(19) United States
(12) Patent Application Publication
(10) Pub. No.: US 2023/0266170 A1
(43) Pub. Date: Aug. 24, 2023

(54) SPECTROMETER-LESS SAMPLE ANALYSIS SYSTEM AND METHOD USING HIGH WAVENUMBER RAMAN SCATTERING

(71) Applicant: CytoVeris Inc., Farmington, CT (US)

(72) Inventors: Alan Kersey, South Glastonbury, CT (US); David Fournier, Northborough, MA (US); Rishikesh Pandey, Unionville, CT (US); Gary Root, Cheshire, CT (US)

(21) Appl. No.: 18/017,812

(22) PCT Filed: Jul. 26, 2021
(86) PCT No.: PCT/US2021/043196

§ 371 Date: Jan. 24, 2023

Related U.S. Application Data
(60) Provisional application No. 63/055,987, filed on Jul. 24, 2020.

(51) Int. Cl. G01J 3/44; G01J 3/12; G01J 3/02; G01J 3/08; G01J 3/10

(52) U.S. Cl. CPC G01J 3/44; G01J 3/12; G01J 3/0208; G01J 3/08; G01J 3/10; G01J 3/0218; G01J 2003/102; G01J 2003/1213; G01N 21/65

(57) ABSTRACT

A system and method for analyzing a sample using Raman spectral light includes a light source, a light detector, a narrow band pass filter and an analyzer. Within the system, excitation light is directed to interrogate the sample. The narrow band pass filter is positioned to receive Raman scattered light produced as a result of the interrogation. The light detector is positioned to receive the Raman scattered light that has passed through the at least one narrow band pass filter. The analyzer contains stored instructions that when executed cause the processor to a) control the light source; and b) process signals produced by the light detector to analyze the sample material, the signals representative of the intensity of the Raman scattered light received by the at least one light detector corresponding to one or more wavenumbers in a high wavenumber region of a Raman signal.

This application claims priority to U.S. Patent Appln. No. 63/055,987 filed Jul. 24, 2020, which is hereby incorporated by reference in its entirety.

### BACKGROUND OF THE INVENTION

For many decades, the reference method for the diagnosis of cancer has been histopathological examination of tissues using conventional microscopy. This process is known as Surgical Pathology. In Surgical Pathology, samples can be produced from surgical procedures (tumor resection), diagnostic biopsies or autopsies. These samples go through a process that includes dissection, fixation, and cutting of tissue into precisely thin slices which are stained for contrast and mounted onto glass slides. The slides are examined by a pathologist under a microscope, and their interpretations of the tissue results in the pathology “read” of the sample.

Advanced optical and electromagnetic (“EM”) imaging approaches have been reported for the determination of tumor margin: These include the use of Fluorescence Imaging [1], Near Infrared spectroscopy [2,3], Raman Spectroscopy [4, 5], and Terahertz reflectivity [6].

Raman spectrum comprises three primary regions of interest: a) the fingerprint region (“FP”) region having wavenumbers typically in the range of about 400 cm⁻¹ to about 1800 cm⁻¹; b) the silent region having wavenumbers typically in the range of about 1800 cm⁻¹ to about 2800 cm⁻¹; and c) the high wavenumber region (“HWN”) region having wavenumbers typically in the range of about 2800 cm⁻¹ to about 3800 cm⁻¹. The FP region typically comprises a series of multiple peaks and is rich in Raman spectral information. In the case of biological samples, the fingerprint region contains information on the content of biomolecular components, such as DNA, proteins, phospholipids, lipids, and the like. Most of the research efforts have only used the FP region for detection of cancerous tissue [7] and the HWN region has largely been underexplored. The relatively underappreciated HWN region typically includes a composite broad spectral shape that includes several underlying peaks associated with the biomolecular content. Recent work in the HWN range has demonstrated successful classification of cancerous tissue versus normal tissue with high sensitivity and specificity [8-10]. The HWN Raman spectral range features reduced unwanted fluorescence and is unaffected by glass as well as tissue marking dye Raman spectra. In addition, it has been shown that combining information from the FP and HWN regions can lead to enhanced performance for some applications [11, 12].

To date, measurement of these specific wavenumber characteristics typically requires a “conventional spectrometer” based system. These systems are often complex and expensive. The present disclosure provides an improved alternative to prior art systems.

### SUMMARY

According to an aspect of the present disclosure, a system for analyzing a sample material using Raman spectral light is provided. The system includes at least one light source, at least one light detector, at least on narrow band pass filter, and an analyzer. The at least one light source is configured to produce excitation light at one or more wavelengths. The system is configured such that excitation light produced by the light source is directed to the sample material to interrogate the sample material, and the at least one narrow band pass filter is positioned to receive Raman scattered light produced as a result of the excitation light interrogation, and the at least one detector is positioned to receive the Raman scattered light that has passed through the at least one narrow band pass filter. The analyzer is in communication with the light source and the at least one light detector and a memory storing instructions. The instructions when executed cause the processor to a) control the light source to produce excitation light at the one or more wavelengths; and b) process signals produced by the light detector to analyze the sample material, the signals representative of the intensity of the Raman scattered light received by the at least one light detector corresponding to one or more wavenumbers in a high wavenumber region of a Raman spectrum.

In any of the aspects or embodiments described above and herein, the at least one light detector may include “N” number of the light detectors, where “N” is an integer equal to or greater than two, and the at least one narrow band pass filter may include “N” number of the narrow band pass filters, and the system may include an “N” way optical splitter device configured to split the received Raman scattered light into “N” paths. The system may be configured such that the optical splitter device is positioned to receive the Raman scattered light and is configured to split the received Raman scattered light into “N” paths, and a respective one of the “N” number of said light detectors and a respective one of the “N” number of said narrow band pass detectors is positioned in a respective one of the “N” paths. The system may be configured such that the split amount of Raman scattered light in each respective path passes through the respective said narrow band pass filter and is received by the respective said light detector.

In any of the aspects or embodiments described above and herein, “N” may equal four.

In any of the aspects or embodiments described above and herein, the system may further include a wavelength controller configured to tune an output of the light source relative to a single said excitation wavelength.

In any of the aspects or embodiments described above and herein, each of the “N” number of narrow band pass filters may be centered on a respective one of the wavenumbers, and the respective one of the wavenumbers of each narrow band pass filter is different than the respective one of wavenumbers of the other narrow band pass filters.

In any of the aspects or embodiments described above and herein, the instructions when executed may cause the processor to process the signals produced by each light detector to produce one or more ratios of the signals representative of the intensity of the Raman scattered light at different respective one of the wavenumbers.

In any of the aspects or embodiments described above and herein, the narrow band pass filters may be configured to have a band pass range of wavelengths that corresponds to a range of 100 cm⁻¹ to 5 cm⁻¹ of said wavenumbers.

In any of the aspects or embodiments described above and herein, the sample material may be a biological tissue sample.

In any of the aspects or embodiments described above and herein, the system may include a wavelength controller configured to selectively cause the light source to produce a plurality of said excitation wavelengths.

In any of the aspects or embodiments described above and herein, the wavelength controller may be in communication with the analyzer, and the instructions when executed may cause the processor to control the wavelength controller to sweep through the plurality of excitation wavelengths.

In any of the aspects or embodiments described above and herein, the at least one light source may include “N” number of light sources where “N” is an integer equal to or greater than two, each light source configured to produce excitation light at a single wavelength, and the single wavelength of excitation light for each light source is different than the single wavelength of excitation light produced by the others of the light sources, and the system may further include an optical switch configured to selectively cause the excitation light from one of the light sources to be passed to the sample material, and a demultiplexer disposed to receive and configured to demultiplex said signals produced by the light detector.

In any of the aspects or embodiments described above and herein, the at least one light source includes “N” number of light sources where “N” is an integer equal to or greater than two, each light source configured to produce excitation light at a single wavelength, and the single wavelength of excitation light for each light source is different than the single wavelength of excitation light produced by the others of the light sources, and the system further may further include an optical combiner configured to combine the excitation light from all of the light sources to form a combined beam of excitation light, and a demultiplexer disposed to receive and configured to demultiplex said signals produced by the light detector.

In any of the aspects or embodiments described above and herein, each light source may be driven by a discrete frequency, and the discrete frequency used to drive each respective light source is different than the discrete frequency used to drive the other respective light sources, and the demultiplexer may be configured to demultiplex the signals produced by the light detector using synchronous detection at each respective discrete frequency.

In any of the aspects or embodiments described above and herein, each light source may be driven by a digital code, and the digital code used to drive each respective light source is different than the digital code used to drive the other respective light sources, and the demultiplexer is configured to demultiplex said signals produced by the light detector using synchronous detection at each respective digital code.

In any of the aspects or embodiments described above and herein, the at least one narrow band pass filter may be tunable and in communication with the analyzer, and the instructions when executed cause the processor to control tunable narrow band pass filter.

In any of the aspects or embodiments described above and herein, the system may include a probe configured to include one or more light conduits for passage of excitation light to the sample material, and for passage of Raman scattered light collected at the sample material.

According to another aspect of the present disclosure, a method for analyzing a sample material using Raman spectral light is provided. The method includes a) interrogating a sample material with excitation light at one or more wavelengths, the excitation light produced by at least one light source; b) filtering Raman scattered light produced by the interrogation using at least one narrow band pass filter; c) detecting the Raman scattering light after said Raman scattering light has passed through the narrow band pass filter using at least one light detector, and producing signals representative of an intensity of the detected Raman scattering light using the at least one detector; and d) processing the signals to analyze the sample material, said processing using the detected intensity of the Raman scattering light at one or more wavenumbers in a high wavenumber region of a Raman spectrum.

In any of the aspects or embodiments described above and herein, the excitation light may be produced by one light source, and the at least one narrow band pass filter may include “N” number of narrow band pass filters, wherein “N” is an integer equal to or greater than two, and the at least one light detector may include “N” number of light detectors, and the method may further include the step of splitting the Raman scattered light produced by the interrogation into “N” paths, and the filtering step may include filtering the split Raman scattered light in each of the “N” paths using a respective one of the narrow band pass filters, and the detecting step may include detecting the split Raman scattered light in each of the “N” paths using a respective one of the light detectors.

In any of the aspects or embodiments described above and herein, the method may further include tuning an output of the light source relative to a single excitation wavelength using a wavelength controller.

In any of the aspects or embodiments described above and herein, each of the “N” number of narrow band pass filters may be centered on a respective one of the wavenumbers, and the respective one of the wavenumbers of each narrow band pass filter is different than the respective one of the wavenumbers of the other narrow band pass filters.

In any of the aspects or embodiments described above and herein, the processing step may include producing one or more ratios of the signals representative of the intensity of the Raman scattered light at different respective one of the wavenumbers.

In any of the aspects or embodiments described above and herein, the step of interrogating the sample material with excitation light may include interrogating the sample material at a plurality of wavelengths of excitation light produced by a single light source.

In any of the aspects or embodiments described above and herein, the step of interrogating the sample material may include sweeping through the plurality of excitation wavelengths.

In any of the aspects or embodiments described above and herein, the step of interrogating the sample material with excitation light at one or more wavelengths produced by at least one light source, may include interrogating the sample material with excitation light at “N” wavelengths, where “N” is an integer equal to or greater than two, using “N” number of light sources, wherein each of the “N” wavelengths is different than the other of the “N” wavelengths, and the method may further include switching the excitation light passed to the sample material between said “N” light sources, and demultiplexing the signals produced by the light detector.

In any of the aspects or embodiments described above and herein, the step of interrogating the sample material with excitation light at one or more wavelengths produced by at least one light source, may include interrogating the sample material with excitation light at “N” wavelengths, where “N” is an integer equal to or greater than two, using “N” number of light sources, wherein each of the “N” wavelengths is different than the other of the “N” wavelengths, and the method may further include combining the excitation light from all of the light sources to form a combined beam of excitation light, and demultiplexing the signals produced by the light detector.

In any of the aspects or embodiments described above and herein, each light source may be driven by a discrete frequency, and the discrete frequency used to drive each respective light source may be different than the discrete frequency used to drive the other respective light sources, and the step of demultiplexing may use synchronous detection at each respective discrete frequency.

In any of the aspects or embodiments described above and herein, each light source may be driven by a digital code, and the digital code used to drive each respective light source may be different than the digital code used to drive the other respective light sources, and the step of demultiplexing may use synchronous detection at each respective digital code.

In any of the aspects or embodiments described above and herein, the at least one light source may be a tunable narrow band pass filter in communication with the analyzer, and the method may further include tuning the tunable narrow band pass filter.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1A is a graph of Raman signal intensity versus wavenumber representative of normal adipose.

FIG. 1B is a graph of Raman signal intensity versus wavenumber representative of fibrous tissue.

FIG. 1C is a graph of Raman signal intensity versus wavenumber representative of diseased cancerous breast tissue.

FIG. 2 is a graph of Raman signal intensity versus wavenumber illustrating the location of well-known underlying sub-peaks associated with HWN Raman spectrum associated with tissue.

FIG. 3 is a diagrammatic representation of a present disclosure system embodiment.

FIG. 4 is a diagrammatic representation of a present disclosure system embodiment.

FIG. 5 is the graph of Raman signal intensity versus wavenumber shown in FIG. 2, now including narrow passband filter function positions in a wavelength or wavenumber domain chosen to align with the underlying un-resolved Raman peaks centered at the wavenumbers indicated by the dashed lines.

FIG. 6 is a diagrammatic representation of a present disclosure system embodiment.

FIG. 7 is a graph of Raman signal intensity versus wavenumber illustrating a HWN peak profile for typical normal adipose and illustrating effective transition of the aforesaid HWN spectrum with offset in light source excitation.

FIG. 8 is a diagrammatic representation of a present disclosure system embodiment.

FIG. 9 is a diagrammatic representation of a present disclosure system embodiment.

FIG. 10 is a diagrammatic representation of a present disclosure system embodiment that includes a probe.

### DETAILED DESCRIPTION

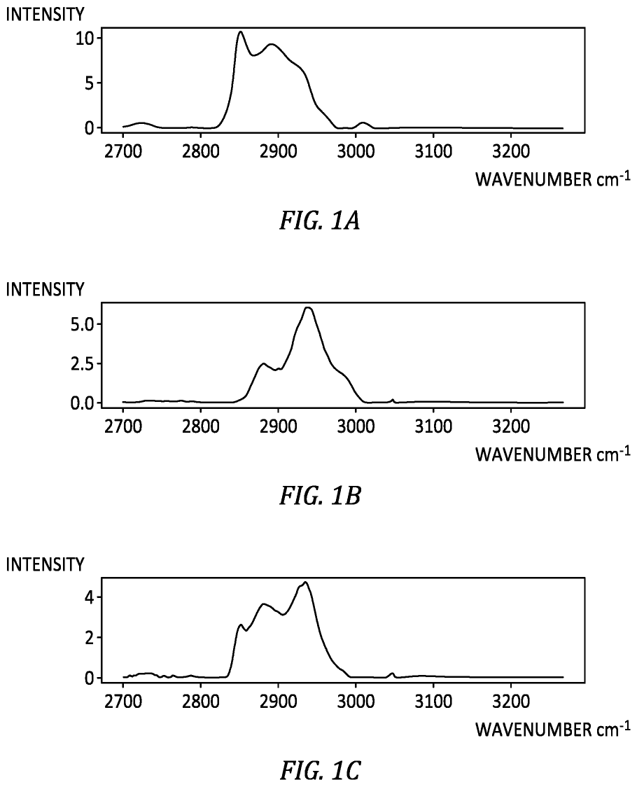

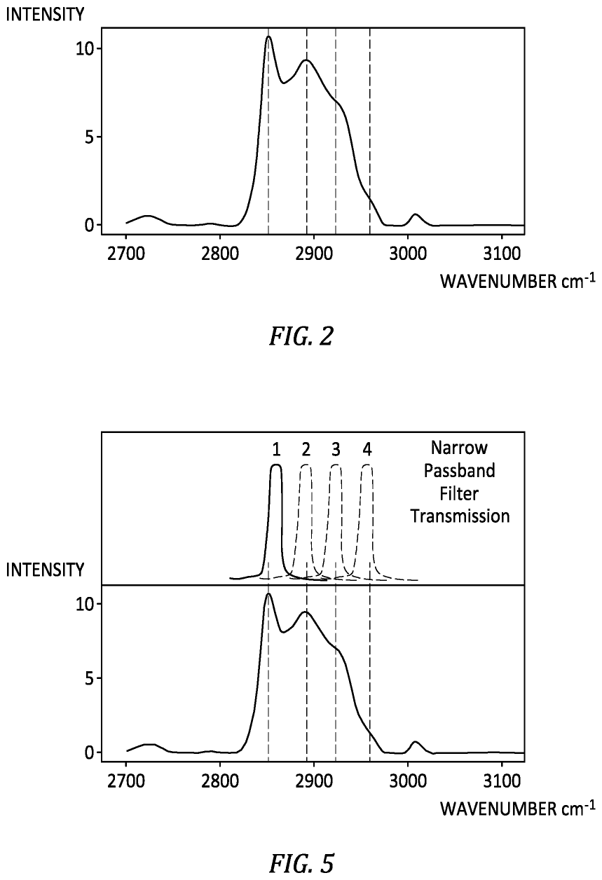

FIGS. 1A-1C illustrate the typical HWN Raman spectrum observed from the analysis of normal adipose (See FIG. 1A), fibrous tissue (See FIG. 1B), and diseased cancerous breast tissue (See FIG. 1C). The HWN spectral profile shapes differ between tissue types, and it has been shown that the tissue type can be identified (also referred to as “classified”) by a measurement of the peak height at a series of points (wavenumber positions) over the HWN intensity profile. FIG. 2 illustrates the location of well-known underlying sub-peaks associated in the HWN Raman spectrum associated with tissue.

Experimental data has indicated ratios of intensity measurements at predetermined wavenumber values can be used to form a “barcoding” approach for identifying the HWN profile shape of a material, which in turn can be used to classifying the material. For example, we have discovered that wavenumbers 2851 cm⁻¹, 2892 cm⁻¹, 2938 cm⁻¹, and 3008 cm⁻¹ can be used to produce six (6) different intensity measurement ratios (e.g., 2851/2892; 2851/2938; 2851/3008; 2892/2938; 2892/3008; and 2938/3008) [13]. An intensity measurement at a wavenumber value of about 2851 cm⁻¹ is understood to reflect C—H vibrations of CH₂ and is further understood to be primarily attributable to the tissue lipid content. An intensity measurement at a wavenumber value of about 2938 cm⁻¹ is understood to reflect C—H vibrations of the CH₃ group and are further understood to be primarily attributable to the tissue protein content. An intensity measurement at a wavenumber value of about 2892 cm⁻¹ is understood to reflect a CH₂ asymmetric stretch of both proteins and lipids within the tissue. An intensity measurement at a wavenumber value of about 3008 cm⁻¹ is understood to reflect the ═C—H vibration associated with lipids and fatty acids that may be present within the tissue. Ratiometric values as described above can be used to create an identifier (i.e., a “barcode”) that can be used to identify an HWN profile shape and to classify a tissue type. This ratiometric aspect of the present disclosure also produces useful parameter values representative of relative tissue constituent contents (e.g., the tissue protein content relative to the tissue lipid content, etc.) and is useful to mitigate variances that may occur (e.g., variances in base line intensity, etc.). Hence, the ratiometric parameters utilized within the present disclosure facilitate tissue type classification. It should be noted that the aforesaid wavenumber values represent examples of wavenumber values that may have significance in certain tissue analysis applications. Other wavenumber values may have significance in alternative tissue analysis applications. Furthermore, the specific wavenumber values provided above represent a wavenumber associated with a HWN peak. These aforesaid wavenumber positions identified in FIG. 2 are representative for breast tissues. Alternative wavenumber values in close proximity to the aforesaid peak wavenumbers may be used for breast tissue analysis purposes. Hence, the present disclosure is not limited to these particular wavenumber values for breast tissue analysis, and other wavenumber values may have significance in tissue analysis. The wavenumber positions identified in FIG. 2 of the present disclosure have been described above in connection with breast tissue. To be clear, the aforesaid wavenumber positions are an example and not a limitation on the scope of the present disclosure. Specific wavenumber details are given in the above description to provide a thorough understanding of the embodiment. This “barcoding” approach is not limited to an analysis of breast tissue and can be used for other tissue types such as but not limited to cervical tissue, bladder tissue, liver tissues, etc. Embodiments of the present disclosure can be used to analyze properties and disease state of a variety of different tissue types such as nonalcoholic fatty liver disease (NAFLD) tissue. The present disclosure may also be used to investigate and analyze healthy tissue types such as muscle tissue present in a transurethral resection of a bladder tumor. In addition, the present disclosure is not limited to analyzing tissue specimens. Embodiments of the present disclosure may be used to analyze other type of biospecimens, including cells, blood or body fluid, metabolites, etc., as well as non-biological materials; e.g., pharmaceutical products, pathogens, chemical products, food products, etc. The present disclosure provides a novel and unobvious method and system operable to rapidly measure specific HWN characteristics of a HWN profile, to generate HWN barcodes using a spectrometer-free detection approach, and to facilitate analysis (e.g., classification) of a biospecimen. Alternative wavenumber values within HWN regions may be used for analysis purposes of other tissue types, biospecimens and other materials and this approach is not limited to the wavenumber positions shown in FIG. 2. The present disclosure is not limited to the analysis of tissues and cells in the ex-vivo/in vitro conditions but could also be used in in-vivo settings including in minimally invasive and robotic surgery.

Aspects of the present disclosure include a system 20 and method for analyzing a biological sample and other materials. To facilitate the description herein, the present disclosure is described in the context of a biological sample. However, as stated above embodiments of the present disclosure system 20 and methods described herein may be used to non-invasively examine a variety of materials. The system 20 includes at least one light source, at least one light detector, at least one narrow passband filter, and an analyzer. Non-limiting exemplary embodiments of the present disclosure system 20 are described herein in greater detail. Unless otherwise indicated, the one or more light sources, the at least one light detector, and analyzer described below may be used in each of the example present disclosure system embodiments described herein.

The one or more light sources are configured to emit coherent light at wavelengths that are useful in Raman spectroscopy. An example of an acceptable coherent light source is a laser. The present disclosure is not limited to using any particular type of laser, or lasers at all. Examples of laser types that may be used include solid state, gas, diode laser or vertical-cavity surface-emitting lasers (VCSELs). The light source is not limited to coherent light at any particular wavelength or wavelength band, but as indicated above coherent light at wavelengths that are useful in Raman spectroscopy are preferred. The operation of the one or more light sources may be controlled by the analyzer.

The at least one light detector is configured to receive Raman scattered light from the interrogated tissue and produce signals representative thereof. The light detector is in signal communication with the analyzer to permit the transfer of signals produced by the light detector to the analyzer. The light detector may be configured to detect Raman scattered light within an acceptable range of wavelengths and intensities for the purposes described herein. The light detector may be chosen to provide optimal performance at one or more wavelengths of light, and at low light intensity levels. Non-limiting examples of light detectors include light detectors that convert light energy into an electrical signal such as a simple photodiode. A specific non-limiting example of a light detector that may be used is an avalanche photodiode. The present disclosure is not limited to any particular type of light detector. In some embodiments, the light intensity sensed by a light detector may be integrated for a time duration “T” to increase the effective signal to noise ratio (SNR).

As will be described herein, embodiments of the present disclosure may include optical elements such as lenses, filters, dichroic mirrors, and the like for processing the excitation light and Raman scattered light. Optical elements such as photonic filters may be used to permit passage of Raman light within one or more defined bandwidths, or to limit optical interference from non-Raman scattered light, or to block excitation light, or any combination thereof from the detection path. The present disclosure is not limited to any particular type of photonic filter and may use more than one type of photonic filter. Non-limiting examples of acceptable photonic tunable filters include colloidal crystal arrays, liquid crystals, acousto-optic tunable filters (AOTF), Fabry Perot, electro-optic, and the like. In some embodiments of the present disclosure, one or more narrow band pass filters may be included that are individually configured to attenuate the received Raman scattered light outside of a predetermined narrow range. In most present disclosure embodiments, the narrow band pass filters are configured to have a band pass range of wavelengths that correspond to a range of 100 cm⁻¹ to 5 cm⁻¹ wavenumbers. More typically, present disclosure narrow band pass filters are configured to have a band pass range of wavelengths that correspond to a range of 80 cm⁻¹ to 20 cm⁻¹ wavenumbers.

The analyzer is in communication with other components within the system 20, such as the one or more light sources, the at least one light detector, and the like to control and or receive signals therefrom to perform the functions described herein. The analyzer may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system to accomplish the same algorithmically and/or coordination of system components. The analyzer may include one or more memory devices and is not limited to any particular type of memory device. The memory device may include read-only memory, random access memory, volatile memory, non-volatile memory, static memory, dynamic memory, flash memory, cache memory, and/or any device that stores digital information. The analyzer may include, or may be in communication with, an input device that enables a user to enter data and/or instructions, and may include, or be in communication with, an output device configured, for example to display information (e.g., a visual display or a printer), or to transfer data, etc. Communications between the analyzer and other system components may be via a hardwire connection or via a wireless connection.

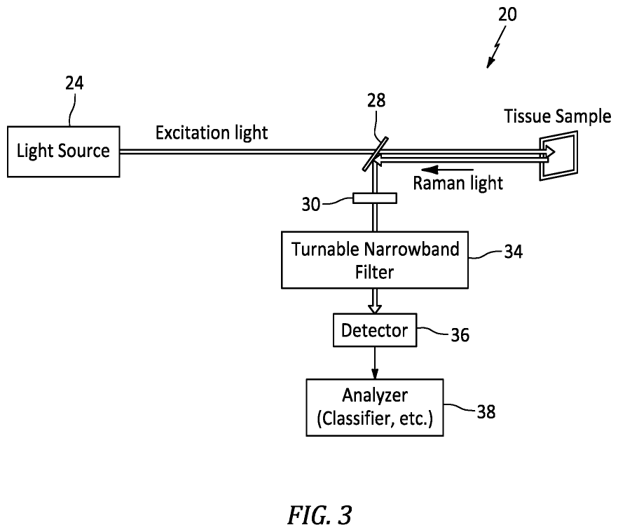

An embodiment of a present disclosure system 20 that utilizes a form of hyperspectral detection is diagrammatically shown in FIG. 3. This system embodiment includes a light source 24, a dichroic mirror 28, an excitation light blocking filter 30, a tunable narrow band pass filter 34, a light detector 36, and an analyzer 38. Non-limiting examples of acceptable tunable narrow band pass filters include colloidal crystal arrays, liquid crystals, acousto-optic tunable filters (AOTF), Fabry-Perot, electro-optic, incident angle dependent filters, and the like. The light source 24 is configured to produce excitation light at a single wavelength. The tunable narrow band pass filter 34 is controllable (e.g., in communication with the analyzer 38) to be tuned (e.g., in a sequential manner) at desired wavenumber positions; e.g., to reflect/transmit different Raman scattered light frequencies.

In the operation of a system embodiment as shown in FIG. 3, the light source 24 is controlled to produce light at a predetermined wavelength. The produced light is passed through the dichroic mirror 28 and is incident to the biological sample being analyzed. Raman scattered light received from the sample as a result of the incident excitation light encounters the dichroic mirror 28 and is directed to pass through the tunable narrow band pass filter 34 prior to encountering the light detector 36. The tunable narrow band pass filter 34 is controlled to reflect/transmit different Raman scattered light frequencies. The Raman light passes through the tunable narrow band pass filter 34 and passes to the light detector 36. The light detector 36 is configured to produce signals representative of the received light and those signals are communicated to the analyzer 38. The analyzer 38 in turn is configured (e.g., through stored executable instructions) to produce information based on the received signals that may be used to analyze the sample being interrogated (e.g., classify the sample). For example, the analyzer may utilize the intensity measurements at predetermined wavenumber values alone, or may use ratiometric values determined using the intensity measurements alone, or any combination thereof directly or indirectly to produce identifying information (i.e., information sufficient to identify the tissue type being interrogated—a “barcode”) that can be used to classify or otherwise analyze the sample tissue. The manner in which the identifying information can be used to classify or otherwise analyze the sample tissue may vary; i.e., the present disclosure is not limited to any particular analysis method. An example of how the identifying information can be used to classify or otherwise analyze the sample tissue includes the use of empirical database stored within the analyzer (or otherwise accessed) that permits a comparative analysis. For example, the empirical database may have clinically sufficient data associated with a variety of different tissue types (e.g., adipose, fibrous tissue, benign tissue, cancerous tissue, etc.) to permit a comparative identification. As another example, the stored instructions within the analyzer may include an algorithmic solution into which identifying information (e.g., intensity values and/or ratiometric values) may be input to permit a tissue type analysis; e.g., a multiparametric methodology. As yet another example, the stored instructions within the analyzer may include a trained classifier that includes a plurality of data sets that permit evaluation of the identifying information and identification of the tissue type. The present disclosure is not limited to any of these exemplary methodologies.

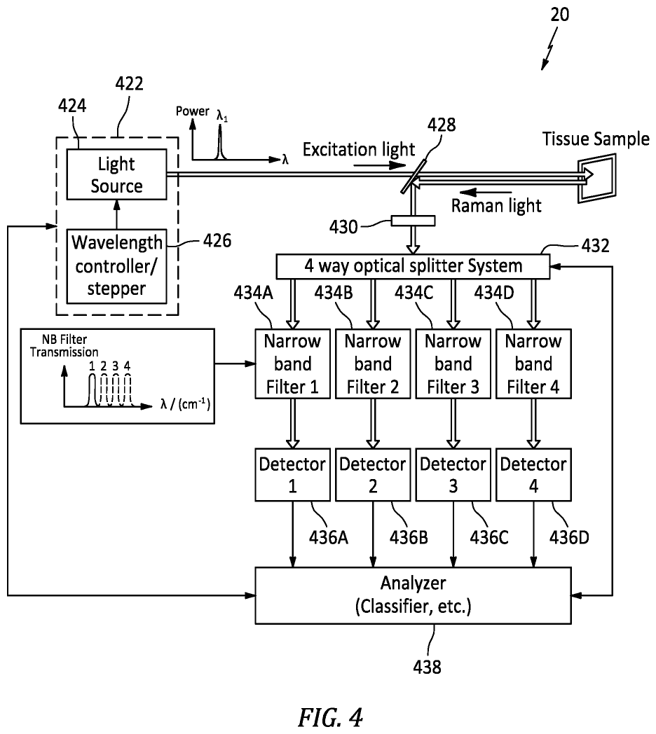

An embodiment of a present disclosure system 20 that utilizes a form of hyperspectral detection is diagrammatically shown in FIG. 4. This system 20 is configured to produce a plurality of light intensity measurements that can be used in the analysis of a sample. Embodiments of the present system 20 allow an HWN profile to be optically “sampled” at a plurality of discrete wavelengths (e.g., four (4)) that correspond to wavenumber locations of the HWN peaks as shown in FIG. 5. This system 20 embodiment includes a light source system 422 (e.g., including a laser 424 and may include a wavelength controller 426), a dichroic mirror 428, an excitation light blocking filter 430, an optical splitter system 432, a plurality of narrow band pass filters 434A-434D, a plurality of light detectors 436A-436D, and an analyzer 438. The optical splitter system 432 used in this system embodiment is configured to split Raman scattered light received from the sample four (4) ways. The split light is directed through four (4) separate narrow band pass filters 434A-434D and subsequently to four (4) separate light detectors 436A-436D. Each of the narrow band pass filters 434A-434D may be centered at a wavelength corresponding to a different wavenumber peak of the HWN Raman signature being analyzed; e.g., see FIG. 5. Signals representative of the light received by each light detector 436A-436D may be communicated to the analyzer 438. The analyzer 438 in turn is configured (e.g., through stored executable instructions) to produce information that may be used to analyze the sample being interrogated; e.g., classify the sample. The present disclosure is not limited to the specific system embodiment diagrammatically shown in FIG. 4. Alternative embodiments may utilize an optical splitter system 432 that splits Raman scattered light received from the sample two or more ways and may include corresponding numbers of narrow passband filters and light detectors.

In the operation of a system embodiment as shown in FIG. 4, a wavelength controller 426 may be in communication with the analyzer 438 and may be used to “tune” the light source (e.g., a laser 424) to produce an excitation light wavelength. An excitation wavelength that is useful in producing a desired HWN Raman signature may be selected. The generated excitation light passes through the dichroic mirror 428 and is incident to the biological sample being analyzed. Raman scattered light received from the sample as a result of the incident excitation light encounters the dichroic mirror 428 and is directed to pass through an excitation light blocking filter 430. The portion of the Raman scattered light that passes through the excitation light blocking filter 430 subsequently encounters the optical splitter system 432. The optical splitter system 432 in turn splits the received light into a plurality of different light paths; e.g., four (4) light paths as shown in FIG. 4. The received Raman scattered light in each light path subsequently passes through a respective narrow band pass filter 434A-434D prior to encountering a respective light detector 436A-436D. As stated above, each of the narrow band pass filters 434A-434D may be centered at a wavelength corresponding to a different wavenumber peak of the HWN Raman signature being analyzed. Each respective light detector 436A-436D is configured to produce signals representative of the received light and those signals are communicated to the analyzer 438. The analyzer 438 in turn is configured (e.g., through stored executable instructions) to produce information that may be used to analyze the sample being interrogated (e.g., classify the sample) in the manner described above.

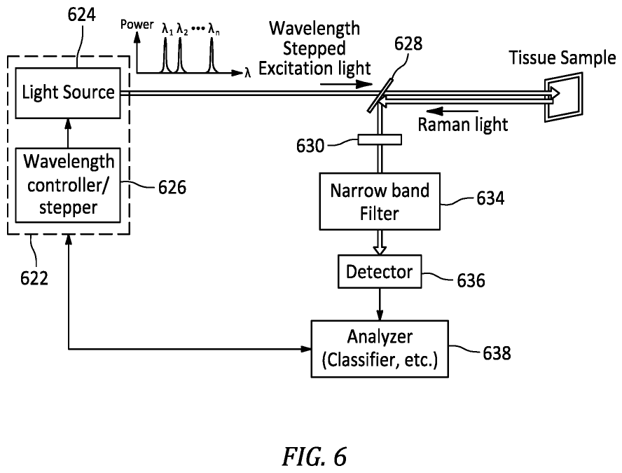

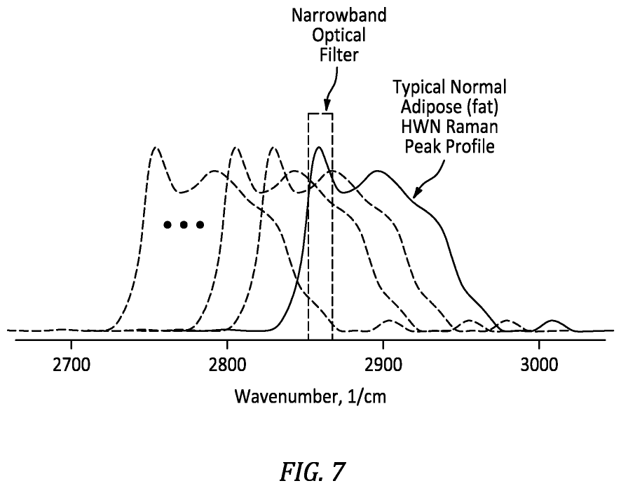

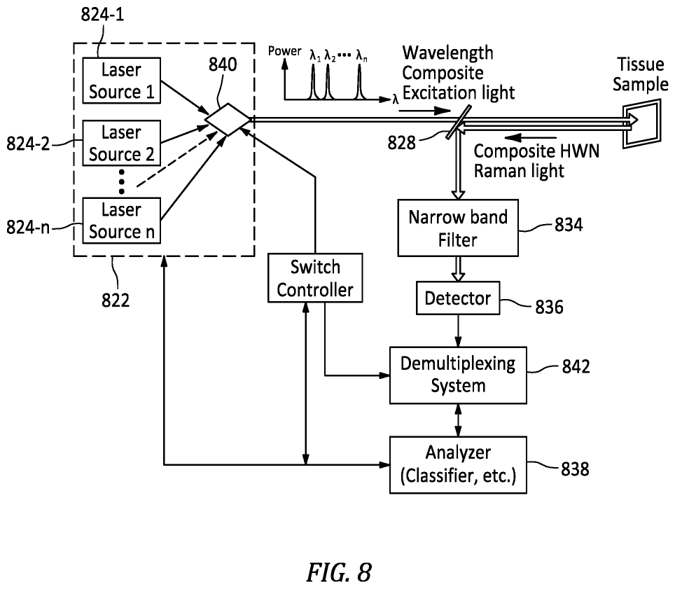

Another embodiment of a present disclosure system 20 that utilizes a form of hyperspectral detection is diagrammatically shown in FIG. 6. This system embodiment includes a light source system 622 (e.g., including a laser 624 and a wavelength controller/stepper 626), a dichroic mirror 628, an excitation blocking filter 630, a narrow band pass filter 634, a light detector 636, and an analyzer 638. In this embodiment, a single detection band may be detected rather than a set of detection wavelength bands being selected as is described herein with respect to FIG. 4. In the system embodiment shown in FIG. 6, the light source (e.g., laser 624) may be controlled to produce excitation light at a plurality of different wavelengths within a predetermined band of wavelengths; i.e., the light source 624 can be “swept” through the aforesaid predetermined band of wavelengths in a sequential manner, thereby permitting signal intensity values of Raman scattered light at different wavenumbers to be detected (e.g., in a sequential manner). FIG. 7 illustrates a graph of the wavenumber peaks that may be sequentially accessed by a system like that shown in FIG. 6.

In the operation of a system embodiment as shown in FIG. 6, the wavelength/controller stepper 626 is in communication with the analyzer 638 and is used to control the light source system 622 to produce excitation light at a plurality of different wavelengths; e.g., “sweeping” through the predetermined band of wavelengths in a sequential manner. The generated excitation light passes through the dichroic mirror 628 and is incident to the biological sample being analyzed. Raman scattered light received from the sample as a result of the incident excitation light encounters the dichroic mirror 628 and is directed to pass through the narrow band pass filter 634 prior to encountering the light detector 636. The light detector 536 is configured to produce signals representative of the received Raman light and those signals are communicated to the analyzer 638. The analyzer 638 in turn is configured (e.g., through stored executable instructions) to produce information that may be used to analyze the sample being interrogated (e.g., classify the sample) in the manner described above.

Another embodiment of a present disclosure system 20 that utilizes a form of hyperspectral detection is diagrammatically shown in FIG. 8. This system embodiment includes a light source system 822, a dichroic mirror 828, a narrow band pass filter 834, a light detector 836, and an analyzer 838. The light source system 822 includes a plurality of independent light sources 824-1, 824-2 . . . 824-n (where “n” is an integer) that each produce excitation light at a different wavelength and an optical switch 840. This system embodiment may include an independent demultiplexing system 842, or the analyzer 838 may be configured to perform demultiplexing.

In the operation of a system embodiment as shown in FIG. 8, the independent light sources (e.g., lasers 824-1, 824-2 . . . 824-n) within the light source system 822 are operated in coordination with the optical switch 840 to produce excitation light at a plurality of different wavelengths. The generated excitation light (at a plurality of different wavelengths) passes through the dichroic mirror 828 and is incident to the biological sample being analyzed. Raman scattered light received from the sample as a result of the incident excitation light encounters the dichroic mirror 828 and is directed to pass through the narrow band pass filter 834 prior to encountering the light detector 836. The light detector 836 is configured to produce signals representative of the received Raman light and those signals are communicated to the independent demultiplexing system 842, or alternatively to the analyzer 838 which may be configured with a demultiplexing capability. The aforesaid signals from the light detector 836 may be time-demultiplexed in a synchronous detection approach. The analyzer 838 in turn is configured (e.g., through stored executable instructions) to produce information based on the demultiplexed signals that may be used to analyze the sample being interrogated (e.g., classify the sample) in the manner described above.

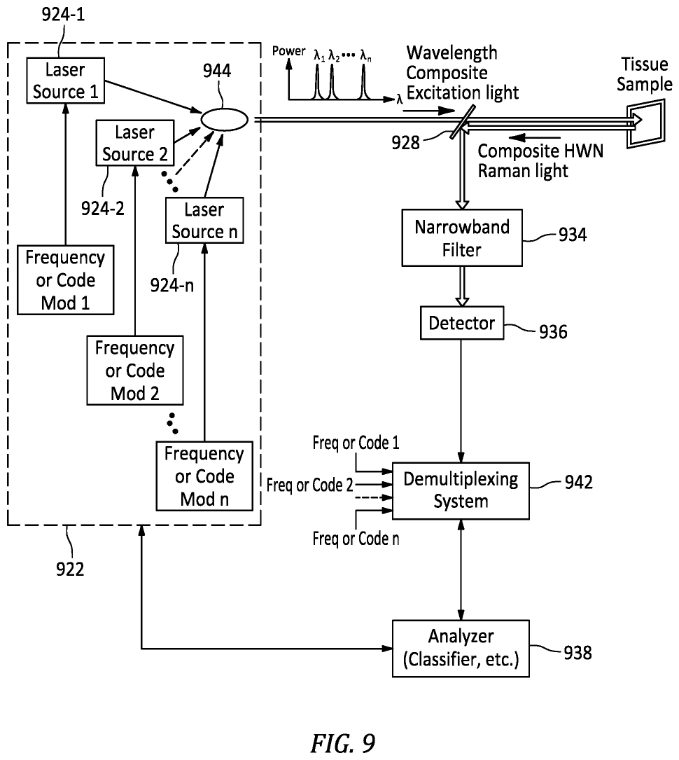

Another embodiment of a present disclosure system 20 that utilizes a form of hyperspectral detection is diagrammatically shown in FIG. 9. This system embodiment includes a light source system 922, a dichroic mirror 928, a narrow band pass filter 934, a light detector 936, and an analyzer 938. The light source system 922 includes a plurality of independent light sources (e.g., lasers 924-1, 924-2 . . . 924-n) that each produce excitation light at a different wavelength and an optical combiner 944. Each of the independent light sources 924-1, 924-2 . . . 924-n within the light source system 922 is configured to produce light at a predetermined distinct wavelength (i.e., each laser produces light at a wavelength different from that produced by the other lasers) and each is driven by a certain discreate frequency (“FDM”) or a digital code, such as a pseudorandom sequence (“CDM”). This system embodiment may include an independent demultiplexing system 942, or the analyzer 938 may be configured to perform demultiplexing.

In the operation of a system embodiment as shown in FIG. 9, each of the independent light sources (e.g., lasers 924-1, 924-2 . . . 924-n) within the light source system 922 is configured to produce light at a predetermined distinct wavelength (i.e., each laser produces light at a wavelength different from that produced by the other lasers) and each is driven by a certain discrete frequency (“FDM”) or a digital code, such as a pseudorandom sequence (“CDM”), or the like. The light produced by the independent light sources 924-1, 924-2 . . . 924-n is passed to the optical combiner 944, and the optical combiner 944 in turn combines the respective laser outputs into a composite beam of light. The composite beam of light passes through the dichroic mirror 928 and is incident to the biological sample being analyzed. Raman scattered light received from the sample as a result of the incident excitation light encounters the dichroic mirror 928 and is directed to pass through the narrow band pass filter 934 prior to encountering the light detector 936. The light detector 936 is configured to produce signals representative of the received Raman light and those signals are communicated to the independent demultiplexing system 942, or alternatively to the analyzer 938 which may be configured with a demultiplexing capability. The Raman HWN responses (i.e., Raman scattered light received from the sample) associated with each excitation wavelength may be offset and overlaid in wavenumber space, but due to the frequency or code modulation of the independent light sources (e.g., lasers 924-1, 924-2 . . . 924-n), the portion of the HWN spectrum passing through the narrow band pass filter 934 (and subsequently detected by the light detector 936) can be demultiplexed by use of synchronous detection at each frequency or code, or the like. The aforesaid signals from the light detector 934 may be time-demultiplexed in a synchronous detection approach. The analyzer 938 in turn is configured (e.g., through stored executable instructions) to produce information based on the demultiplexed signals that may be used to analyze the sample being interrogated (e.g., classify the sample) in the manner described above.

As indicated above, the present disclosure is described above primarily in the context of a system 20 and method for examining a biological sample; e.g., a breast tissue sample. Embodiments of the present disclosure are not, however, limited to examining biological samples. The present disclosure system 20 and methods described above may be used to non-invasively examine a variety of materials (e.g., pharmaceutical products, chemical products, food products, etc.) Hence, in applications for analysis of materials (regardless of the material type), the HWN profiles may represent constituents normally present within the material under analysis.

As is clear from the description above, the present disclosure provides a system and methodology for analyzing materials (e.g., classification of tissue types) that obviates the need for a spectrometer. The present disclosure leverages the Raman HWN spectral range using one or more narrow band pass filters to “sample” the HWN profile of the material being analyzed at meaningful points on the HWN profile. As described above, the present disclosure permits sample analysis using determined Raman scattered light intensity values, or ratiometric values based on those intensity values, or some combination thereof. The analysis (e.g., classification) of the sample may utilize an empirical database within a comparative analysis, or an algorithmic/multiparametric approach into which identifying information (e.g., intensity values and/or ratiometric values) may be utilized, or a trained classifier that permits evaluation using a plurality of data sets, or the like. In addition, while spontaneous Raman spectra have been used in the present disclosure (e.g., FIGS. 1A-1C) to illustrate the present disclosure, the present disclosure is not limited to using spontaneous Raman spectra. In alternative embodiments, the analysis/use of the HWN region described herein may be derived from alternative variants of Raman spectroscopy such as stimulated Raman spectroscopy, resonance Raman spectroscopy, surface enhanced Raman spectroscopy, and the like. Still further, the present disclosure is not limited to using any particular excitation wavelength or any wavelength region.

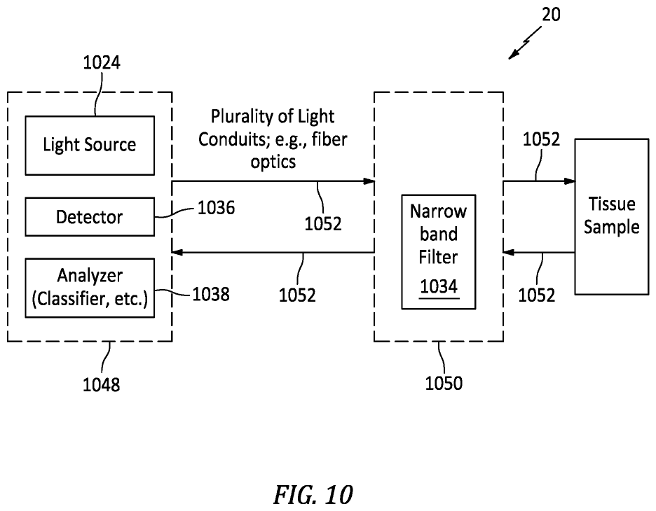

Aspects of the present disclosure are described herein in terms of system embodiments. The system embodiments are described and shown diagrammatically. The present disclosure system embodiments may be configured to analyze ex-vivo samples, or in the case of biospecimen applications the system embodiments may be configured to permit in-vivo analyses. For example, the aforesaid systems may be configured as a probe (e.g., the entirety of the system is configured as a probe) or may be configured to include a probe that can be used for in-vivo sample interrogation. The probe may be configured to deliver the excitation light and to collect the Raman scattering light. FIG. 10 diagrammatically illustrates a system 20 embodiment wherein the light source(s) 1024, the light detector 1036, and the analyzer 1038 are disposed in an instrument 1048 that is configured for use with a probe 1050 that may be used for in-vivo examination. The probe 1050 is in communication with the instrument 1048 via light conduits (e.g., optical fibers 1052). In the embodiment shown in FIG. 10, the narrow band pass filter is shown as a portion of the probe 1050; e.g., some of the fiber optics 1052 that are configured to collect the Raman scattered light may have tips modified to function as narrow band pass filters (diagrammatically shown as filter 1034). The embodiment diagrammatically shown in FIG. 10 is a non-limiting example. In alternative embodiments, various system components may be located in one or the other of the probe 1050 or instrument 1048. The aforesaid system 20 embodiments that include a probe 1050 are understood to provide significant utility during minimally invasive procedures, including but not limited to endoscopic procedures and robotic surgical applications. and may be used in many

[symbol]

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details. For example, the system embodiment examples provided herein are non-limiting examples of the present disclosure system. Each of the exemplary systems are described as including certain optical elements (e.g., filters, etc.). The disclosure of these optical elements within the respective exemplary system embodiments should not be construed as these optical elements being required. In alternative embodiments, some described optical elements may be omitted or exchanged with different optical elements that provide the same similar functionality, and additional optical elements may be included.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

Additionally, even though some features, concepts, or aspects of the disclosures may be described herein as being a preferred arrangement or method, such description is not intended to suggest that such feature is required or necessary unless expressly so stated. Still further, exemplary, or representative values and ranges may be included to assist in understanding the present application, however, such values and ranges are not to be construed in a limiting sense and are intended to be critical values or ranges only if so expressly stated.

### REFERENCES

1. Nguyen and Tsien, “Fluorescence-guided surgery with live molecular navigation—a new cutting edge”, Nat Rev Cancer, 13(9), pp. 653-662,2013.
2. Tummers, et al., “Real-time intraoperative detection of breast cancer using near-infrared fluorescence imaging and methylene blue”, Eur J Surg Oncol., 40(7), 850-858,2014.
3. Dahr et al., “A diffuse reflectance spectral imaging system for tumor margin assessment using custom annular photodiode arrays”, Biomedical Optics Express, 3, (12), 2012.
4. Pence I., Mahadevan-Jansen A., “Clinical instrumentation and applications of Raman spectroscopy”, Chem Soc Rev.; 45 (7):1958-1979, 2016.
5. Talari, A. et al., “Raman Spectroscopy of Biological Tissues”, Applied Spectroscopy Reviews, 50:1, 46-111, 2015.
6. Yaroslaysky. A, et al., “Delineating nonmelanoma skin cancer margins using terahertz and optical imaging”, J of Biomedical Photonics & Eng., 3(1), 2017.
7. A. S. Haka, et al., “In vivo margin assessment during partial mastectomy breast surgery using Raman spectroscopy,” Cancer Res. 66(6), 3317-3322 (2006).
8. J. Mo, et al., “High wavenumber Raman spectroscopy for in vivo detection of cervical dysplasia,” Anal. Chem. 81(21), 8908-8915 (2009).
9. K. Lin, et al., “Optical diagnosis of laryngeal cancer using high wavenumber Raman spectroscopy,” Biosens. Bioelectron. 35(1), 213-217 (2012).
10. PCT Publication No. WO 2004/051242, “Use of High Wavenumber Raman Spectroscopy for Measuring Tissue”, Puppels, Gerwin.
11. Aubertin et al., “Combining high wavenumber and fingerprint Raman spectroscopy for the detection of prostate cancer during radical prostatectomy”, 9, (9), BIOMEDICAL OPTICS EXPRESS, p. 4294, (2018)
12. Wang et al., “Simultaneous fingerprint and high-wavenumber fiber-optic Raman spectroscopy improves in vivo diagnosis of esophageal squamous cell carcinoma at endoscopy, Scientific Reports, 5, Article number: 12957 (2015)
13. R. Pandey et al, “High Wavenumber Raman Barcoding for Cancer tissue Classification”, CytoVeris pending publication.

1. A system for analyzing a sample material using Raman spectral light, comprising:
at least one light source configured to produce excitation light at one or more wavelengths;
at least one light detector;
at least one narrow band pass filter;
wherein the system is configured such that excitation light produced by the light source is directed to the sample material to interrogate the sample material, and the at least one narrow band pass filter is positioned to receive Raman scattered light produced as a result of the excitation light interrogation, and the at least one detector is positioned to receive the Raman scattered light that has passed through the at least one narrow band pass filter; and
an analyzer in communication with the light source and the at least one light detector and a memory storing instructions, which instructions when executed cause the analyzer to:
control the light source to produce excitation light at the one or more wavelengths; and
process signals produced by the light detector to analyze the sample material, the signals representative of an intensity of the Raman scattered light received by the at least one light detector corresponding to one or more wavenumbers in a high wavenumber region of a Raman spectrum.

2. The system of claim 1, wherein the at least one light detector includes “N” number of said light detectors, where “N” is an integer equal to or greater than two, and the at least one narrow band pass filter includes “N” number of said narrow band pass filters, and the system further comprises an “N” way optical splitter device configured to split the received Raman scattered light into “N” paths; and
wherein the system is configured such that the optical splitter device is positioned to receive the Raman scattered light and is configured to split the received Raman scattered light into “N” paths, and a respective one of the “N” number of said light detectors and a respective one of the “N” number of said narrow band pass detectors is positioned in a respective one of the “N” paths, and the system is configured such that the split amount of Raman scattered light in each respective path passes through the respective said narrow band pass filter and is received by the respective said light detector.

3. The system of claim 2, wherein “N” equals four.

4. The system of claim 2, wherein the system further comprises a wavelength controller configured to tune an output of the light source relative to a single said excitation wavelength.

5. The system of claim 2, wherein each of the “N” number of said narrow band pass filters is centered on a respective one of said wavenumbers, and the respective one of said wavenumbers of each said narrow band pass filter is different than the respective one of said wavenumbers of the other said narrow band pass filters.

6. The system of claim 5, wherein the instructions when executed cause the analyzer to process the signals produced by each said light detector to produce one or more ratios of the signals representative of the intensity of the Raman scattered light at different respective said one of said wavenumbers.

7. The system of claim 2, wherein the narrow band pass filters are configured to have a band pass range of wavelengths that corresponds to a range of 100 cm⁻¹ to 5 cm⁻¹ of said wavenumbers.

8. The system of claim 2, wherein the narrow band pass filters are configured to have a band pass range of wavelengths that corresponds to a range of 80 cm⁻¹ to 20 cm⁻¹ of said wavenumbers.

9. The system of claim 1, wherein the sample material is a biological tissue sample.

10. The system of claim 1, further comprising a wavelength controller configured to selectively cause said light source to produce a plurality of said excitation wavelengths.

11. The system of claim 10, wherein the wavelength controller is in communication with the analyzer; and
wherein the instructions when executed cause the analyzer to control the wavelength controller to sweep through the plurality of excitation wavelengths.

12. The system of claim 1, wherein the at least one light source includes “N” number of light sources where “N” is an integer equal to or greater than two, each said light source configured to produce said excitation light at a single wavelength, and said single wavelength of excitation light for each light source is different than the single wavelength of excitation light produced by the others of the light sources; and
wherein the system further includes an optical switch configured to selectively cause the excitation light from one of the light sources to be passed to the sample material, and a demultiplexer disposed to receive and configured to demultiplex said signals produced by the light detector.

13. The system of claim 1, wherein the at least one light source includes “N” number of light sources where “N” is an integer equal to or greater than two, each said light source configured to produce said excitation light at a single wavelength, and said single wavelength of excitation light for each light source is different than the single wavelength of excitation light produced by the others of the light sources; and
wherein the system further includes an optical combiner configured to combine the excitation light from all of the light sources to form a combined beam of excitation light, and a demultiplexer disposed to receive and configured to demultiplex said signals produced by the light detector.

14. The system of claim 13, wherein each light source is driven by a discrete frequency, and the discrete frequency used to drive each respective light source is different than the discrete frequency used to drive the other respective light sources, and the demultiplexer is configured to demultiplex said signals produced by the light detector using synchronous detection at each respective discrete frequency.

15. The system of claim 13, wherein each light source is driven by a digital code, and the digital code used to drive each respective light source is different than the digital code used to drive the other respective light sources, and the demultiplexer is configured to demultiplex said signals produced by the light detector using synchronous detection at each respective digital code.

16. The system of claim 1, wherein the at least one narrow band pass filter is tunable and is in communication with the analyzer, and the instructions when executed cause the analyzer to control tunable narrow band pass filter.

17. The system of claim 1, further comprising a probe configured to include one or more light conduits for passage of said excitation light to the sample material, and for passage of said Raman scattered light collected at said sample material.

18. A method for analyzing a sample material using Raman spectral light, comprising:
interrogating a sample material with excitation light at one or more wavelengths, the excitation light produced by at least one light source;
filtering Raman scattered light produced by the interrogation using at least one narrow band pass filter;
detecting the Raman scattering light after said Raman scattering light has passed through the narrow band pass filter using at least one light detector, and producing signals representative of an intensity of the detected Raman scattering light using the at least one detector; and
processing the signals to analyze the sample material, said processing using the detected intensity of the Raman scattering light at one or more wavenumbers in a high wavenumber region of a Raman spectrum.

19. The method of claim 18, wherein the excitation light is produced by one light source, and the at least one narrow band pass filter includes “N” number of narrow band pass filters, wherein “N” is an integer equal to or greater than two, and the at least one light detector includes “N” number of light detectors; and
the method further comprising the step of splitting the Raman scattered light produced by the interrogation into “N” paths; and
the filtering step includes filtering said split Raman scattered light in each of the “N” paths using a respective one of the narrow band pass filters; and
the detecting step includes detecting said split Raman scattered light in each of the “N” paths using a respective one of the light detectors.

20. The method of claim 19, wherein “N” equals four.

21. The method of claim 19, further comprising tuning an output of the light source relative to a single said excitation wavelength using a wavelength controller.

22. The method of claim 19, wherein each of the “N” number of said narrow band pass filters is centered on a respective one of said wavenumbers, and the respective one of said wavenumbers of each said narrow band pass filter is different than the respective one of said wavenumbers of the other said narrow band pass filters.

23. The method of claim 22, wherein the processing step includes producing one or more ratios of the signals representative of the intensity of the Raman scattered light at different respective said one of said wavenumbers.

24. The method of claim 19, wherein the narrow band pass filters are configured to have a band pass range of wavelengths that corresponds to a range of 100 cm⁻¹ to 5 cm⁻¹ of said wavenumbers.

25. The method of claim 18, wherein the sample material is a biological tissue sample.

26. The method of claim 18, wherein the step of interrogating the sample material with excitation light includes interrogating the sample material at a plurality of wavelengths of excitation light produced by a single said light source.

27. The method of claim 26, wherein the step of interrogating the sample material includes sweeping through the plurality of excitation wavelengths.

28. The method of claim 18, wherein the step of interrogating the sample material with excitation light at one or more wavelengths produced by at least one light source, includes interrogating the sample material with excitation light at “N” wavelengths, where “N” is an integer equal to or greater than two, using “N” number of light sources, wherein each of said “N” wavelengths is different than the other of said “N” wavelengths; and
the method further comprising switching the excitation light passed to the sample material between said “N” light sources; and
demultiplexing the signals produced by the light detector.

29. The method of claim 18, wherein the step of interrogating the sample material with excitation light at one or more wavelengths produced by at least one light source, includes interrogating the sample material with excitation light at “N” wavelengths, where “N” is an integer equal to or greater than two, using “N” number of light sources, wherein each of said “N” wavelengths is different than the other of said “N” wavelengths; and
the method further includes combining the excitation light from all of the light sources to form a combined beam of excitation light; and
demultiplexing the signals produced by the light detector.

30. The method of claim 18, wherein each light source is driven by a discrete frequency, and the discrete frequency used to drive each respective light source is different than the discrete frequency used to drive the other respective light sources; and
the step of demultiplexing uses synchronous detection at each respective discrete frequency.

31. The method of claim 18, wherein each light source is driven by a digital code, and the digital code used to drive each respective light source is different than the digital code used to drive the other respective light sources; and
the step of demultiplexing uses synchronous detection at each respective digital code.

32. The method of claim 18, wherein the at least one light source is a tunable narrow band pass filter; and
the method further comprises tuning the tunable narrow band pass filter.
